(19) United States
(12) Patent Application Publication
(10) Pub. No.: US 2026/0048027 A1
(43) Pub. Date: Feb. 19, 2026

(54) METHYLATED PHLORETIN ANALOGS AS INHIBITORS OF SGLT2

(71) Applicant: SOCIÉTÉ DES PRODUITS NESTLÉ S.A., Vevey (CH)

(72) Inventors: Yann RATINAUD, Morges (CH); Robin WILLOWS, Echandens (CH); Matthew SANDERS, Epalinges (CH); Olivier CICLET, Lonay (CH); Marine KRAUS, Vuarrens (CH)

(21) Appl. No.: 19/102,944

(22) PCT Filed: Aug. 14, 2023
(86) PCT No.: PCT/EP2023/072381

§ 371 Date: Feb. 11, 2025

(30) Foreign Application Priority Data
Aug. 18, 2022 (EP) 22191049.0

(51) Int. Cl. A61K 31/12; A61K 36/73; A61K 36/752

(52) U.S. Cl. CPC A61K 31/12; A61K 36/73; A61K 36/752

(57) ABSTRACT

The present invention relates to compositions and methods for improving metabolic health. Specifically, the present disclosure relates to using a methylated phloretin analog as a sodium-glucose transporter-2 (SGLT2) inhibitor for improving metabolic health.

### CROSS REFERENCE TO RELATED APPLICATIONS

The present application is a National Stage of International Application No. PCT/EP2023/072381 filed Aug. 14, 2023, which claims priority to European Patent Application No. 22191049.0 filed Aug. 18, 2022, the entire contents of which are incorporated herein by reference.

### BACKGROUND

The present disclosure generally relates to compositions and methods for improving metabolic health. Specifically, the present disclosure relates to using a methylated phloretin analog as a sodium-glucose transporter-2 (SGLT2) inhibitor for improving metabolic health. For example, it was found for the first time that methylated phloretin analogs can be used both as a food or supplement and as a sodium-glucose transporter-2 (SGLT2) inhibitor to effectively inhibit SGLT2, thus improving metabolic health.

The SGLT family of proteins include active glucose transporters that play an important role in maintaining glucose balance in the blood. SGLT2 is expressed in the kidneys on the epithelial cells lining the first segment of the proximal convoluted tubule and is responsible for approximately 90% of the kidney's glucose reabsorption. By inhibiting SGLT2, an ideal inhibitor would prevent the reuptake of glucose from the glomerular filtrate in the kidney and subsequently lower the glucose level in the blood and promote the excretion of glucose in the urine.

Phlorizin is a natural compound and known inhibitor of SGLT1 and SGLT2. SGLT1 is expressed in the gut in addition to the kidney. Inhibition of SGLT1 in the gut is associated with negative gastrointestinal effects including diarrhea. Phlorizin is associated with negative gastrointestinal effect and cannot be added to food as a supplement and is not approved for use as a medicine. The present invention does not result in SGLT1 inhibition in the gut and is therefore different in this way to phlorizin and could therefore be added to food.

The known SGLT2 inhibitors are an insulin-independent class of oral antihyperglycemic medication that clinicians use in the treatment of type 2 diabetes. There are four SGLT2 inhibitors approved by the Food and Drug Administration since 2013: canagliflozin, dapagliflozin, empagliflozin and ertugliflozin.

The known SGLT2 inhibitors are a class of prescription medicines that are FDA-approved for use with diet and exercise to lower blood sugar in adults with type 2 diabetes. Medicines in the SGLT2 inhibitor class include canagliflozin, dapagliflozin, and empagliflozin. They are available as single-ingredient products and also in combination with other diabetes medicines such as metformin. SGLT2 inhibitors lower blood sugar by causing the kidneys to remove sugar from the body through the urine. However, none of these known SGLT2 inhibitors are natural compounds and they can only be use as a drug and not as a food or supplements.

There is a clear unmet need for new compounds and compositions as SGLT2 inhibitors for improving metabolic health. Needed in the art is new compounds and compositions as SGLT2 inhibitors, which can be used as a food or supplement for improving metabolic health.

### SUMMARY

The present disclosure includes the recognition that administering a subject a compound of a methylated phloretin analog or related compositions as a sodium-glucose transporter-2 (SGLT2) inhibitor can effectively inhibit SGLT2, thus improving metabolic health.

The methylated phloretin analog is selected from the group consisting of Phloretin-4-methyl-ether, 4′-O-Methylphloretin, Calomelanone and Flavokavain A.

The methylated phloretin analog or related compositions have an improved bioavailability compared to phloretin and can be used as a food or supplement to a subject to effectively inhibit SGLT2, thus improving metabolic health.

Additionally, such compound and related compositions have a decreased potency (2 to 3 fold) on GLUT inhibition.

Another advantage of the present compound and related compositions is an improved bioconversion since fewer glycosides can be generated in vivo compared to phloretin.

### BRIEF DESCRIPTION OF DRAWINGS

FIG. 1 (A-E) show level of incorporated [3H]2-deoxyglucose, a GLUT transporter substrate following treatment with (A) Phloretin, (B) Calomelanone, (C) Phloretin 4-methyl ether, (D) Asebogenin, (E) Flavokavain A. Data is shown relative to the effect of the pharmacological GLUT inhibitor BAY-876 (100% effect) and DMSO (0% effect).

FIG. 2-5(A-D): Incubations of specified individual methyl-phloretin compounds: Calomelanone (FIG. 2); Phloretin-4 methyl ether (FIG. 3); Phloretin-4′ methyl ether (Asebogenin) (FIG. 4); Flavokavain (FIG. 5) with UDP-glucose in the presence of rat microsomes were performed and analyzed by uHPLC-MS following solid phase extraction as described. For each compound:
i) Extracted mass chromatograms are shown for the expected monoisotopic mass of the parental and glycoside forms from an LC-MS analysis of an SPE eluate fraction containing both parent and generated glycoside metabolites (A).
ii) For each peak observed in the extracted mass chromatograms for parent and glycoside species, a summation of the MS level spectra obtained over the peak duration is displayed (more than 5 individual detection events), to which the Waters Lockmass correction has been applied for the determination of the accurate monoisotopic mass (B).
iii) For each major peak for parent and derived glycoside species observed in the extracted mass chromatograms, an MS/MS spectra for the selected parent ion and glycoside species is shown (C). For the glycoside species, the selected ions fragment with a characteristic loss of 162 mu consistent with loss of a glucosyl moiety, producing fragmentation ions at the expected m/z of the parent methyl-phloretin compound.
iv) For each methylphloretin species, the position of expected non-equivalent sites of glycosylation are indicated (D).

FIG. 6 is a graph showing normalized SGLT1 and SGLT2 inhibition activities of 4′ methyl phlorizin (NI00046322) and chemical formula.

FIG. 7 is a graph showing normalized SGLT1 and SGLT2 inhibition activities of 4 methyl phlorizin (NI00046291) & (NI00001380) and chemical formula.

FIG. 8 represents chemical formula of the methylated phloretin analogs and glycosides corresponding to the lettering shown in table 1.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “at least one dithionite or a functionally similar reducing agent” should be interpreted as “dithionite,” or “a functionally similar reducing agent,” or “both dithionite and a functionally similar reducing agent.”

Where used herein, the terms “example” and “such as,” particularly when followed by a listing of terms, are merely exemplary and illustrative and should not be deemed to be exclusive or comprehensive. As used herein, a condition “associated with” or “linked with” another condition, means the conditions occur concurrently, preferably means that the conditions are caused by the same underlying condition, and most preferably means that one of the identified conditions is caused by the other identified condition.

The term “a sodium-glucose transporter-2 inhibitor,” or “SGLT2 inhibitor,” as used herein, refers to a compound or substance which exhibits an inhibitory effect on sodium-sugar carboxy-2 (SGLT2), in particular on human SGLT2. The inhibitory effect on hSGLT2 measured by IC50 is preferably 1,000 nM or less, more preferably 100 nM or less, and most preferably 50 nM or less. The IC50 value of an SGLT2 inhibitor is typically greater than 0.01 nM, or even greater than 0.1 nM. The inhibitory effect on hSGLT2 can be measured by methods known in the literature, in particular as described in application WO 2005/092877 or WO 2007/093610 (page 23/24); is hereby incorporated by reference. The term “SGLT2 inhibitor” also includes the pharmaceutically acceptable salts, hydrates and solvates thereof, including the respective crystal forms.

In one embodiment, the known SGLT2 inhibitor is selected from the group consisting of dapagliflozin, canagliflozin, empagliflozin, artigliprozine, resmogliflozin, sergliprozine and their derivatives.

For example, the known SGLT2 inhibitor may include a class of prescription medicines that are FDA-approved for use with diet and exercise to lower blood sugar in adults with type 2 diabetes. The known medicines in the SGLT2 inhibitor class may include canagliflozin, dapagliflozin, and empagliflozin. They are available as single-ingredient products and also in combination with other diabetes medicines such as metformin. SGLT2 inhibitors lower blood sugar by causing the kidneys to remove sugar from the body through the urine.

Curr Opin Endocrinol Diabetes Obes. 2017 February; 24(1): 73-79. doi: 10.1097/MED to Hsia et al provides a list of other compounds as the SGLT2 inhibitor.

However, none of these known SGLT2 inhibitors are natural compounds and they can only be use as a drug and not as a food or supplements.

The term “nutritional composition,” or “nutritional supplement,” as used herein, refers to a nutritional product that provides nutrients to an individual that may otherwise not be consumed in sufficient quantities by the individual. For instance, a nutritional composition or nutritional supplement of the present invention may include vitamins, minerals, fiber, fatty acids, or amino acids. Nutritional compositions or nutritional supplements of the present invention may for example be provided in the form of a pill, a tablet, a lozenge, a chewy capsule or tablet, a tablet or capsule, or a powder supplement that can for example be dissolved in water or sprinkled on food.

In one embodiment, nutritional compositions or nutritional supplements of the present invention may provide selected nutrients while not representing a significant portion of the overall nutritional needs of a subject. Typically, they do not represent more than 0.1%, 1%, 5%, 10% or 20% of the daily energy need of a subject. A nutritional composition or nutritional supplement of the present invention may be used in any subject, such as a subject during pregnancy, e.g., as a maternal supplement.

As used herein, an “effective amount,” or “pharmaceutically effective amount,” as used herein, refers to an amount that prevents a deficiency, treats a disease or medical condition in an individual or, more generally, reduces symptoms, manages progression of the diseases or provides a nutritional, physiological, or medical benefit to the individual.

The term “unit dosage form,” as used herein, refers to physically discrete units suitable as unitary dosages for human and animal subjects, each unit containing a predetermined quantity of the nutritional composition disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of powder in a sachet.

The term “nutritional product,” as used herein, refers to any product that can be used to provide nutrition to a subject. Typically, a nutritional product contains a protein source, a carbohydrate source and a lipid source.

The term “food product,” as used herein, refers to any kind of product that may be safely consumed by a human or an animal. A food product may be in solid, semi-solid or liquid form and may comprise one or more nutrients, foods or nutritional supplements. For instance, the food product may additionally comprise the following nutrients and micronutrients: a source of proteins, a source of lipids, a source of carbohydrates, vitamins and minerals. The food product may also contain anti-oxidants, stabilizers (when provided in solid form) or emulsifiers (when provided in liquid form).

The term “functional food product,” as used herein, refers to a food product providing an additional health-promoting or disease-preventing function to the individual.

The term “healthy ageing product,” as used herein, refers to a product providing an additional health-promoting or disease-preventing function related to healthy ageing to the individual.

The term “dairy products,” as used herein, refers to food products produced from milk or fractions of milk from animals such as cows, goats, sheep, yaks, horses, camels, and other mammals. Examples of dairy products are low fat milk (e.g., 0.1%, 0.5% or 1.5% fat), fat-free milk, milk powder, whole milk, whole milk products, butter, buttermilk, buttermilk products, skim milk, skim milk products, high milk-fat products, condensed milk, creme fraiche, cheese, ice cream and confectionery products, probiotic drinks or probiotic yoghurt type drinks.

The term “dairy alternative product,” as used herein, refers to products similar to dairy products but produced without milk.

The term “milk,” as used herein, is defined by Codex Alimentarius as the normal mammary secretion of milking animals obtained from one or more milkings without either addition to it or extraction from it, intended for consumption as liquid milk or for further processing.

The term “beverage product,” as used herein, refers to a nutritional product in liquid or semi-liquid form that may be safely consumed by an individual.

The term “diet product,” as used herein, refers to a food product with a restricted and/or reduced caloric content.

The term “pet food product,” as used herein, refers to a nutritional product that is intended for consumption by pets. A pet, or companion animal, as referenced herein, is to be understood as an animal selected from dogs, cats, birds, fish, rodents such as mice, rats.

As used herein, an “effective amount” is an amount that prevents a deficiency, treats a disease or medical condition in an individual or, more generally, reduces symptoms, manages progression of the diseases or provides a nutritional, physiological, or medical benefit to the individual.

All ingredients of the composition can be admixed together or alternatively the composition can be provided in the form of a kit of parts wherein ingredients or groups of ingredients are provided separately. These separate compositions may be intended to be consumed separately or together.

The term “analog” as used herein, refers to a compound having a structure similar to that of another one, but differing from it in respect of a certain component. A “derivative” is a compound that can be imagined to arise or is actually be synthesized from a parent compound by replacement of one or more atoms with another atom or group of atoms.

TABLE 1
List of phloretin analogs, common names and expected
monoisotopic exact masses for the [M − H]−
ion of the aglycone and glycoside species. Chemical
formula of Table 1 compound are shown in FIG. 8.

| | | | Expected | |
|---|---|---|---|---|
| | | | monoisotopic | Observed |
| | | | mass [M − H] | [M − H] |
| | Description | Common Name | ion | ion masses |
| A | phloretin | Phloretin | 273.0763 | 273.0770 |
| B | methyl- | Phloretin-4 methyl ether | 287.092 | 287.0918 |
| | phloretin | | | |
| C | methyl- | Phloretin-4′ methyl | 287.092 | 287.0920 |
| | phloretin | ether, Asebogenin | | |
| D | dimethyl- | Calomelanone | 301.1076 | 301.1086 |
| | phloretin | | | |
| E | trimethyl- | Flavokavain-A | 315.1232 | 315.1233 |
| | phloretin | | | |
| F | phloretin- | Phlorizin | 435.1291 | 435.1289, |
| | glycoside | | | 435.1287, |
| | | | | 435.1289 |
| G | methyl- | Phloretin-4 methyl ether | 449.1448 | 449.1449, |
| | phloretin- | glycoside | | 449.1450 |
| | glycoside | | | |
| H | methyl- | Phloretin-4′ methyl | 449.1448 | 449.1454, |
| | phloretin- | ether glycoside | | 449.1452 |
| | glycoside | | | |
| I | dimethyl- | Calomelanone glycoside | 463.1604 | 463.1617 |
| | phloretin- | | | |
| | glycoside | | | |
| J | trimethyl- | Flavokavain-A glycoside | 477.1761 | 477.1764 |
| | phloretin- | | | |
| | glycoside | | | |

https://www.molbase.com/cas/76172-68-4.html
https://www.molbase.com/cas/520-42-3.html

The term “Calomelanone” as used herein, refers to 2′,6′-dihydroxy-4,4′-dimethoxydihydrochalcone, with CAS number of 35241-54-4.

The term “Flavokavain A” as used herein, refers to 2′-Hydroxy-4,4′,6′-trimethoxychalcone with CAS number of 37951-13-6.

The term “Phloretin-4-methylether refers to 1-(2,6-dihydroxy-4-methoxyphenyl)-3-(4-hydroxyphenyl) propan-1-one with CAS number of 520-42-3.

The term “4′-O-methylphloretin refers to Asebogenin or 1-(2,6-dihydroxy-4-methoxyphenyl)-3-(4-hydroxyphenyl) propan-1-one with CAS number of 520-42-3.

It is understood that according to certain embodiments, the compound of the invention or composition thereof may be a nutraceutical composition, pharmaceutical composition, functional food, functional nutrition product, medical food, medical nutrition product, or a dietary supplement.

The terms “nutraceutical” combines the words “nutrition” and “pharmaceutical”. It is a food or food product that provides health and medical benefits, including the prevention and treatment of a condition, disorder, or disease. A nutraceutical is a product isolated or purified from foods that is generally sold in medicinal forms not usually associated with food. A nutraceutical is demonstrated to have a physiological benefit or provide protection against a condition, disorder, or disease. Such products may range from isolated nutrients, dietary supplements and specific diets to genetically engineered foods, herbal products, and processed foods such as cereals, soups, and beverages.

The term “nutraceutical,” as used herein, denotes usefulness in both nutritional and pharmaceutical fields of application. Thus, novel nutraceutical compositions can be used as supplements to food and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellifying agents, gel-forming agents, antioxidants and antimicrobials.

Moreover, a multi-vitamin and mineral supplement may be added to nutraceutical compositions of the invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions of the invention may be in any galenic form that is suitable for administering to the body, especially in any form that is conventional for oral administration, e.g. in solid forms such as food or feed, food or feed premix, fortified food or feed, tablets, pills, granules, dragees, capsules and effervescent formulations such as powders and tablets, or in liquid forms, such as solutions, emulsions or suspensions as e.g. beverages, pastes and oily suspensions. The pastes may be incorporated in hard or soft shell capsules, whereby the capsules feature e.g. a matrix of (fish, swine, poultry, cow) gelatine, plant proteins or lignin sulfonate. Examples for other application forms are those for transdermal, parenteral or injectable administration. The dietary and pharmaceutical compositions may be in the form of controlled (delayed) release formulations.

Beverages encompass non-alcoholic and alcoholic drinks as well as liquid preparations to be added to drinking water and liquid food. Non-alcoholic drinks are e.g. soft drinks, sports drinks, fruit juices, teas and milk-based drinks. Liquid foods are e.g. soups and dairy products. The nutraceutical composition comprising the compound of the invention may be added to a soft drink, an energy bar, or a candy.

If the nutraceutical composition is a pharmaceutical formulation and the composition further contains pharmaceutically acceptable excipients, diluents or adjuvants then standard techniques may be used for their formulation, as e.g., disclosed in Remington's Pharmaceutical Sciences, 20th edition Williams & Wilkins, PA, USA. For oral administration, tablets and capsules are preferably used which contain a suitable binding agent, e.g., gelatine or polyvinyl pyrrolidone, a suitable filler, e.g. lactose or starch, a suitable lubricant, e.g. magnesium stearate, and optionally further additives.

The term “functional food,” “functional nutrition product,” “medical food,” or “medical nutrition product,” as used herein, refers to any healthy food claimed to have a health-promoting or disease-preventing property beyond the basic function of supplying nutrients. The general category of functional foods includes processed food or foods fortified with health-promoting additives, like “vitamin-enriched” products.

The terms “food,” “food product” and “food composition” or “diet product” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet.

A dietary supplement, also known as food supplement or nutritional supplement, is a preparation intended to supplement the diet and provide nutrients, such as vitamins, minerals, fibre, fatty acids, or amino acids that may be missing or may not be consumed in sufficient quantities in a person's diet. Some countries define dietary supplements as foods, while in others they are defined as drugs or natural health products. Supplements containing vitamins or dietary minerals are included as a category of food in the Codex Alimentarius, a collection of internationally recognized standards, codes of practice, guidelines and other recommendations relating to foods, food production and food safety. These texts are drawn up by the Codex Alimentarius Commission, an organization that is sponsored by the Food and Agriculture Organization of the United Nations (FAO) and the World Health Organization (WHO).

Compositions intended for an animal, include food compositions to supply the necessary dietary requirements for an animal, animal treats (e.g., biscuits), and/or dietary supplements. The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of the UFA and NORC, and B vitamins and antioxidants. This permits the supplement to be administered to the animal in small amounts, or in the alternative, can be diluted before administration to an animal. The dietary supplement may require admixing, or can be admixed with water or other diluent prior to administration to the animal.

“Pet food” or “pet treat compositions” comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof. The compositions may further comprise from about 5% to about 40% fat. The compositions may further comprise a source of carbohydrate. The compositions may comprise from about 15% to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

As used herein, the term “diabetes” includes insulin-dependent diabetes mellitus (i.e. IDDM, also known as type 1 diabetes) non-insulin-dependent diabetes mellitus (i.e. NIDDM, also known as type 2 diabetes), and prediabetes. Type 1 diabetes is the result of an absolute deficiency of insulin, the hormone which regulates glucose utilization. Type 2 diabetes often occurs in the face of normal, or even elevated levels of insulin and appears to be the result of the inability of tissues to respond appropriately to insulin. This is termed “insulin resistance”. Most type 2 diabetic patients are also overweight or obese. One of the criteria for diagnosing diabetes is the fasting plasma glucose level. A diabetic subject has a fasting plasma glucose level of greater than or equal to 126 mg/dl. A prediabetic subject is someone suffering from prediabetes. A prediabetic subject is a subject with impaired fasting glucose (a fasting plasma glucose level of greater than or equal to 100 mg/dl and less than 126 mg/dl); or impaired glucose tolerance (a 2-hour plasma glucose level of ≥140 mg/dl and <200 mg/dl); or insulin resistance, resulting in an increased risk of developing diabetes. Prevention of type 2 diabetes includes treatment of prediabetes.

As used herein, the term “dyslipidemia” encompasses abnormal levels of any lipid fractions as well as specific lipoprotein abnormalities. For example, it refers to elevation of plasma cholesterol and/or elevation of triglycerides and/or elevation of free fatty acids and/or low high-density lipoprotein (HDL) level and/or high low-density lipoprotein (LDL) level and/or high very low-density lipoprotein (VLDL) level. Dyslipidemia may for example contribute to the development of atherosclerosis and ultimately symptomatic vascular disease including coronary heart disease. Dyslipidemia may or may not be associated with diabetes.

As used herein, the term “metabolic disorder” encompasses any abnormal chemical and enzymatic reactions disrupting normal metabolism due to environmental and genetic factors (environmental factors include physical activity, nutrition), leading to excessive levels or deficiency of certain substances and dysfunction of energy homeostasis. Non-limiting examples of metabolic disorders include diabetes, dyslipidemia, hypertension, being overweight, obesity, and any combination thereof.

The term “prevention” or “preventing,” as used herein, refers to reduction of risk and/or severity of a condition, disorder, or disease.

The term “treatment,” “treating,” “treat,” “attenuate,” or “alleviate,” as used herein, refers to both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder, and include treatment of patients at risk of contracting a disease or suspected to have contracted a disease, as well as patients who are ill or have been diagnosed as suffering from a disease or medical condition. The term does not necessarily imply that a subject is treated until total recovery. These terms also refer to the maintenance and/or promotion of health in a subject not suffering from a disease but who may be susceptible to the development of an unhealthy condition. These terms are also intended to include the potentiation or otherwise enhancement of one or more primary prophylactic or therapeutic measure. The terms “treatment,” “treat,” “attenuate” and “alleviate” are further intended to include the dietary management of a disease or condition or the dietary management for prophylaxis or prevention a disease or condition. A treatment can be patient- or doctor-related.

Obesity, which is an excess of body fat relative to lean body mass, is a chronic disease that is highly prevalent in modern society. It is associated not only with a social stigma, but also with decreased life span and numerous medical problems, including adverse psychological development, coronary artery disease, hypertension, stroke, diabetes, hyperlipidemia, and some cancers, (see, e.g., Nishina, et al., Metab. 43:554-558, 1994; Grundy and Barnett, Dis. Mon. 36:641-731, 1990; Rissanen, et al., British Medical Journal, 301:835-837, 1990).

The term “obesity related disorders,” as used herein, refers to those diseases or conditions where excessive body weight or high “body mass index (BMI)” has been implicated in the progression or suppression of the disease or condition. Representative examples of obesity related disorders include, without limitation diabetes, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia. See, Harrison's Principles of Internal Medicine, 13th Ed., McGraw Hill Companies Inc., New York (1994). Examples, without limitation, of inflammatory conditions include diseases of the digestive organs (such as ulcerative colitis, Crohn's disease, pancreatitis, gastritis, benign tumor of the digestive organs, digestive polyps, hereditary polyposis syndrome, colon cancer, rectal cancer, stomach cancer and ulcerous diseases of the digestive organs), stenocardia, myocardial infarction, sequelae of stenocardia or myocardial infarction, senile dementia, cerebrovascular dementia, immunological diseases and cancer in general.

The term “subject,” “individual,” or “patient,” as used herein, refers to any animal, including a human, that could benefit from one or more of the compounds, compositions or methods disclosed herein. Generally, the subject is a human or an avian, bovine, canine, equine, feline, hircine, lupine, murine, ovine or porcine animal. The “companion animal,” as used herein, refers to any domesticated animal, and includes, without limitation, cats, dogs, rabbits, guinea pigs, ferrets, hamsters, mice, gerbils, horses, cows, goats, sheep, donkeys, pigs, and the like. Preferably, the subject is a human or a companion animal such as a dog or cat. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly subjects. For other animals, an “older adult” has exceeded 50% of the average lifespan for its particular species and/or breed within a species. An animal is considered “elderly” if it has surpassed 66% of the average expected lifespan, preferably if it has surpassed the 75% of the average expected lifespan, more preferably if it has surpassed 80% of the average expected lifespan. An elderly cat or dog has an age from birth of at least about 7 years.

In one embodiment, the term “subject,” as used herein, refers to a mammal. Mammal includes, but is not limited to, rodents, aquatic mammals, domestic animals such as dogs and cats, farm animals such as sheep, pigs, cows and horses, and humans. In one embodiment, the mammal may be a cat, a dog or a human. The human may be a woman, for example, a woman who is trying to get pregnant, or who is pregnant. In one embodiment of the invention, the subject is a mammal selected from the group consisting of a cat, a dog and, a human. For example, the subject may be an old human.

The term “aglycone,” “aglycon,” or “genin,” as used herein, refers to the compound remaining after the glycosyl group on a glycoside is replaced by a hydrogen atom. For example, the aglycone of a cardiac glycoside would be a steroid molecule.

### Embodiments

### Compounds and Compositions

An aspect of the present disclosure is a compound and a composition comprising such a compound for use as a sodium-glucose transporter-2 (SGLT2) inhibitor.

In one embodiment, the compound and the related composition comprising such a compound can be used both as a food or supplement and as a SGLT2 inhibitor to effectively inhibit SGLT2, thus improving a subject's metabolic health.

Applicant surprisingly found that administering a subject in need thereof a methylated phloretin analog or related compositions as a SGLT2 inhibitor can effectively inhibit SGLT2, thus improving metabolic health. Such compounds have numerous advantages compared to phloretin and SGLT2 inhibitors, and in particular have a decreased potency (2 to 3 fold) on GLUT inhibition so that the side effects of the known SGLT2 inhibitors can be avoided.

It was previously reported that the known SGLT2 inhibitors (e.g., canagliflozin, dapagliflozin, empagliflozin and ertugliflozin) have many side effects. For example, genital infections seem to be the most common adverse effect of these known SGLT2 inhibitors. Other side effects may also include mycotic infections, urinary tract infections and osmotic diuresis, risk of diabetic ketoacidosis (DKA), decreased bone mineral density and therefore increased risk of bone fractures, increased risk of lower limb amputation, or an increased risk of Fournier gangrene.

In one embodiment, the methylated phloretin analogs or related compositions as a SGLT2 inhibitor in the present invention can reduce or remove all these side effects.

Additionally, the methylated phloretin analogs or related compositions have an improved bioavailability compared to phloretin as well as an improved solubility and can be used as a food or supplement to a subject to effectively inhibit SGLT2, thus improving metabolic health.

Another advantage of the present compound and related compositions is an improved bioconversion since fewer glycosides can be generated in vivo compared to phloretin.

In an aspect, the present disclosure relates to a composition for use in the inhibition of SGLT2 in a subject comprising a methylated phloretin analog. The subject is a mammal such as a cat, a dog or a human. In one embodiment, the subject is a female human or a male human. In one preferred embodiment, the subject is an old female human or an old male human.

In one embodiment, the methylated phloretin analog is selected from the group consisting of Phloretin-4-methyl-ether, 4′-O-Methylphloretin, Calomelanone and Flavokavain A.

In one embodiment, the methylated phloretin analog as disclosed herein is for the use to treat or prevent a condition, disorder, or disease related to type 2 diabetes, and/or obesity in a subject.

In one embodiment, the methylated phloretin analog as disclosed herein is for the use to treat or prevent a condition, disorder, or disease related to cardiovascular, weight management, renal, kidney or brain.

In one embodiment, the methylated phloretin analog as disclosed herein is used as a SGLT2 inhibitor through either a direct inhibition mechanism or an indirect inhibition mechanism.

In one embodiment, the methylated phloretin analog as disclosed herein is used as a SGLT2 inhibitor through a direct inhibition mechanism.

In one embodiment, the inhibition of SGLT2 by using the methylated phloretin analog can occur anywhere in the subject.

In one embodiment, the inhibition of SGLT2 by using the methylated phloretin analog as disclosed herein is in muscle and/or kidney tissues.

In one embodiment, the methylated phloretin analog as disclosed herein may be obtained from a plant or plant extract. In a preferred embodiment, it can be obtained from unripe apple e.g. by de-glycosylation followed by methylation. It can also be obtained from orange peel by de-glycosylation.

In one embodiment, the methylated phloretin analog as disclosed herein is for use in the preparation of a medicament for treating or preventing a condition, disorder, or disease responsive to SGLT2 inhibition.

In one aspect, the present invention relates to a composition comprising the methylated phloretin analog as disclosed herein.

For example, the composition comprising the methylated phloretin analog as disclosed herein can be used both as a food or supplement and as an effective SGLT2 inhibitor.

Thus, in one embodiment, the composition is a food, beverage, or dietary supplement comprising the methylated phloretin analog as one of the ingredients.

In another aspect, the present invention relates to a pharmaceutical composition comprising a therapeutically effective amount of the methylated phloretin analog as disclosed herein, or a pharmaceutically acceptable salt or solvate thereof, as active ingredient, and a pharmaceutically acceptable carrier, for use in the inhibition of SGLT2.

It may be administered in accordance with the present invention in any pharmaceutically effective amount. Typically, a pharmaceutically effective amount will depend on the type, age, size, health status, lifestyle and/or genetic heritage of the subject. The pharmaceutically effective amount may be split into several smaller amounts and administered throughout the day so as the total daily intake is the effective amount. A person skilled in the art will be able to propose appropriate amounts of the methylated phloretin analog to be consumed per day.

In one embodiment, the methylated phloretin analog as disclosed herein in the present invention may be provided in an amount of between about 0.001 mg and about 100 mg per daily dose, 0.005 mg and about 50 mg per daily dose, about 0.01 mg and about 20 mg per daily dose, about 0.015 mg and about 10 mg per daily dose.

In one embodiment, the composition comprising the methylated phloretin analog as disclosed herein may be used as a nutritional composition or nutritional supplement. For example, the composition comprising the methylated phloretin analog as disclosed herein, might be beneficial to a human subject (such as an old adult or a patient whose metabolic health needs improvements) for inhibiting SGLT2, thus improving his/her metabolic health.

In one embodiment, the composition comprising the methylated phloretin analog as disclosed herein may be used for daily supplemental or routine administration. For example, it may be administered to an old adult or a patient whose metabolic health needs improvements. In one embodiment, the composition may be administered to a human subject with a metabolic disorder such as obesity, type 2 diabetes, cardiovascular disease or with an obesity related disorder such as diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

In some embodiments, the composition may further comprise at least one of an excipient, a diluent, or a carrier.

Non-limiting examples of suitable excipients may include a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient may be a buffering agent. Non-limiting examples of suitable buffering agents may include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical composition.

In some embodiments, an excipient may comprise a preservative. Non-limiting examples of suitable preservatives may include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In one embodiment, the composition may further comprise a binder. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The composition may be administered by any method appreciated by the skilled artisan. For example, the methylated phloretin analog of the invention or composition thereof is preferably administered by oral administration. In some embodiments, it may be administered by intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

In one embodiment, the composition of the invention may have an acute effect that can be seen in less than one month. Additionally or alternatively, the composition can have a long-term effect, and thus various embodiments comprise administration of the composition to the individual (e.g., orally) for a time period of at least one month; preferably at least two months, more preferably at least three, four, five or six months; most preferably for at least one year. During the time period, the composition can be administered to the individual at least one day per week; preferably at least two days per week, more preferably at least three, four, five or six days per week; most preferably seven days per week. The composition can be administered in a single dose per day or in multiple separate doses per day. In one embodiment, a single dose is not less than about 100 mg. In one embodiment, a single dose is not more than about 1000 mg. In one embodiment, a single dose is between about 100 mg and about 1000 mg.

In one embodiment, the composition may be in a liquid form or in a solid form (e.g., solid dosage forms).

In some embodiments, solid dosage forms of the composition for oral administration may include capsules, tablets, caplets, pills, troches, lozenges, powders, and granules.

For example, a capsule may comprise a core material comprising the composition comprising the methylated phloretin analog and a shell wall that encapsulates a core material. In some embodiments, a core material may comprise at least one of a solid, a liquid, and an emulsion. In some embodiments, a shell wall material may comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments, at least one polymer may function as taste-masking agents.

In some embodiments, tablets, pills, and the like may be compressed, multiply compressed, multiply layered, and/or coated. For example, a coating may be single or multiple.

Liquid formulations of the composition comprising the methylated phloretin analog may include a syrup (for example, an oral formulation), an intravenous formulation, an intranasal formulation, an ocular formulation (e.g., for treating an eye infection), an otic formulation (e.g., for treating an ear infection), an ointment, a cream, an aerosol, and the like. In some instances, a combination of various formulations may be administered. In some embodiments, a tablet, pill, and the like can be formulated for an extended release profile. In some embodiments, a composition may be formulated to increase the shelf stability when stored in a closed container under standard ambient conditions.

Each of the components in the composition of the present invention may be used in any amount that is effective in achieving the objective of the present invention (i.e., increasing breastmilk micronutrient levels of a subject after the subject gives birth). For example, the skilled artisan would be able to determine appropriate dosages depending on age, size and health status of each specific subject, on her lifestyle, as well as on her genetic heritage.

In one embodiment, the amounts used in the present application are amounts per daily dose. The amount of each component may be used as disclosed or changed (e.g., increased or decreased) depending on age, size and health status of each specific subject, on her lifestyle, as well as on her genetic heritage. In one embodiment, the nutritional composition or nutritional supplement of the present invention may be administered regularly, for example two times a day, daily, every two days or weekly.

In an aspect, the composition of the present invention may be in any form that is suitable to administer all the ingredients. For example, the composition of the present invention can be in the form of a powdered nutritional composition to be reconstituted in milk or water, a food product, a drink, a nutritional supplement or a nutraceutical.

When the composition of the present invention is in the form of a powdered nutritional composition to be reconstituted in milk or water, the nutritional composition or nutritional supplement may preferably comprise a protein source, a carbohydrate source and a lipid source, preferably together with lecithin. The composition may also comprise soya lecithin and/or a bulking agent. The protein source may be dried milk or dried skimmed milk. As carbohydrate source sucrose and/or maltodextrin may be used. The lipid source may be vegetable oil. The formulation may also alternatively or additionally contain glucose syrup, milk fat, magnesium citrate, choline salts and esters, prebiotic fibers, and/or ascorbyl palmitate. Flavor compounds, such as cocoa powder or honey, for example, may be added to provide taste variations.

In another aspect, the composition of the present invention may be a product selected from the group consisting of a nutritional product, a functional food product, a healthy ageing product, a dairy product, a dairy alternative product, a beverage product, a diet product, and a pet food product.

In one embodiment, the composition comprising the methylated phloretin analog as disclosed herein may be used for treating or preventing a condition, disorder, or disease related to type 2 diabetes, non-alcoholic fatty liver disease and/or obesity in a subject.

In one embodiment, the condition may be a metabolic disorder such as obesity, type 2 diabetes, cardiovascular disease or an obesity related disorder such as diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

In another embodiment, the composition comprising the methylated phloretin analog as disclosed herein may be used for treating or preventing a condition selected from the group consisting of obesity, type 2 diabetes, cardiovascular disease, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

### Methods

Another aspect of the present disclosure is a method for improving metabolic health (e.g., treating or preventing a condition, disorder, or disease related to type 2 diabetes, and/or obesity, cardiovascular, weight management, renal, kidney or brain, a metabolic disorder or an obesity related disorder) by inhibiting SGLT2. In one embodiment, the method comprises administering a subject in need thereof a composition comprising the methylated phloretin analog of the present invention as a SGLT2 inhibitor.

In one embodiment, the method for improving metabolic health comprises treating or preventing a metabolic disorder. In one embodiment, the metabolic disorder comprises obesity, type 2 diabetes, cardiovascular disease. In one embodiment, the metabolic disorder is selected from the group consisting of obesity, type 2 diabetes, and cardiovascular disease.

In another embodiment, the method for improving metabolic health comprises treating or preventing an obesity related disorder. In one embodiment, the obesity related disorder comprises diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia. In one embodiment, the obesity related disorder is selected from the group consisting of diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

In one embodiment, the present disclosure is a method of treating or preventing a condition selected from the group consisting of obesity, type 2 diabetes, cardiovascular disease, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

In another embodiment, the present disclosure is a method of treating or preventing a condition, disorder, or disease related to type 2 diabetes, non-alcoholic fatty liver disease and/or obesity in a subject by inhibiting SGLT2.

As disclosed above, the method comprises administering a subject in need thereof a composition comprising at least one of Phloretin-4-methyl-ether, 4′-O-Methylphloretin, Calomelanone or Flavokavain A.

In one embodiment, the method comprises administering a subject in need thereof a composition comprising at least Phloretin-4-methyl-ether.

In one embodiment, the method comprises administering a subject in need thereof a composition comprising at least 4′-O-Methylphloretin.

In one embodiment, the method comprises administering a subject in need thereof a composition comprising at least calomelanone.

In one embodiment, the method comprises administering a subject in need thereof a composition comprising at least flavokavain A.

In one embodiment, the method comprises administering a subject in need thereof a composition comprising at least one of Phloretin-4-methyl-ether, 4′-O-Methylphloretin, Calomelanone or Flavokavain A, to inhibit SGLT2.

In one embodiment, the inhibition of SGLT2 is through either a direct inhibition mechanism or an indirect inhibition mechanism.

In one embodiment, the inhibition of SGLT2 is through a direct inhibition mechanism.

In one embodiment, the inhibition of SGLT2 occurs at any tissue in the subject related to the condition, disorder, or disease.

In one embodiment, the inhibition of SGLT2 is in muscle, liver and/or kidney tissues.

In one embodiment, the methylated phloretin analog is obtained from a plant or plant extract.

In one embodiment, the methylated phloretin analog of the present invention can be used to inhibit reabsorption of glucose in the kidney and therefore lower blood sugar.

In one embodiment, binding of different sugars of the compound of the present invention to the glucose site affects the orientation of the aglycone in the access vestibule. Thus, when the aglycone of the compound of the present invention binds it affects the entire inhibitor. Applicant envisions that these mechanisms together might lead to a synergistic interaction. Therefore, variations in the structure of both the sugar and the aglycone of the compound of the present invention are crucial for the pharmacophore of SGLT inhibitors.

In one embodiment, the compound of the present invention and the related composition can lead to at least 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% decrease on glucose level in the subject as compared with the control subjects who were not administered with the methylated phloretin analog of the present invention and the related composition.

In one embodiment, the decrease on glucose level can be sustained up to 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 36 months, or 48 months.

In another aspect, the present invention relates to an in vitro method of inhibiting SGLT2. The method comprises contacting a methylated phloretin analog with SGLT2 and determining inhibition of SGLT2.

In one embodiment, direct inhibition of SGLT2 may be determined by ability to bind to SGLT2.

In yet another aspect, the present invention relates to a method of treatment or prevention of a condition, disorder, or disease related to cardiovascular, weight management, renal, kidney, brain, type 2 diabetes, and/or obesity comprising administration of a composition comprising the methylated phloretin analog as disclosed herein.

### EXAMPLES

### Example 1: Methylated Analogs of Phloretin Exhibit a Reduced Degree of GLUT Inhibition

### Protocol

CHO-K1 cells were maintained at 37° C. with 5% CO2 in DMEM growth media containing 4.5 g/L Glucose, Glutamax, Sodium pyruvate (Thermo, 10569010) supplemented with 10% fetal bovine serum (Thermo, 10270106) supplemmented with non-essential amino acids (Thermo, 11140050). Cells were plated in 24 well tissue culture test plates (TPP, 92424) at 80% confluency. To determine GLUT inhibition, wells were washed 4 times with krebs-ringers-hepes (140 mM NaCl, 4.7 mM KCl, 2.5 mM CaCl2, 1.25 mM MgSO4, 1.2 mM KH2PO4, 10 mM hepes, 2 mM sodium pyruvate, pH 7.4) solution to remove extracellular glucose prior to transfer of 250 μL krebs-ringers-hepes solutions containing vehicle, phloretin or the specified methylphloretin analog at the indicated concentrations, or GLUT inhibitor BAY-876 (Merck, SML1774-5 MG) at 10 μM at 0.1% (v/v) DMSO final for 30 minutes treatment duration. Following the treatment period, 2-deoxyglucose at 100 μM spiked with 10 μCi [3H]2-deoxyglucose (Perkin Elmer, NET328A001MC) was added to the wells for 5 minutes at 37° C. The reaction was stopped via addition of 5 mM glucose to the wells before washing 4 times with krebs-ringers-hepes buffer to remove extracellular [3H]2-deoxyglucose. Cells were lysed with 0.1 M NaOH and the relative level of incorporated radioactivity determined as counts per minute per well measured using a TriCarb scintillation counter with UltimaGold Scintillant (Perkin Elmer, 6013329). All other reagents from Sigma.

### Results

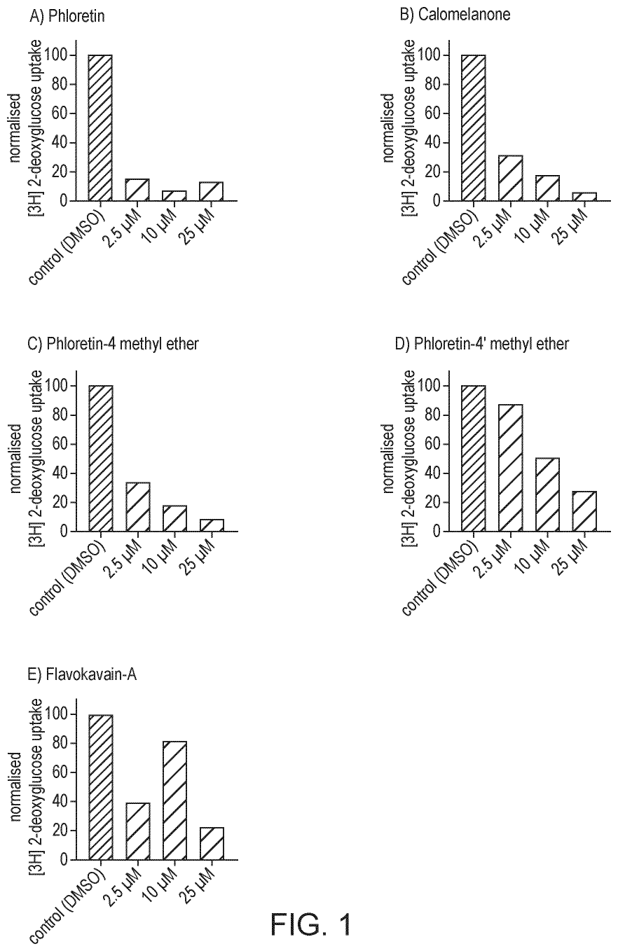

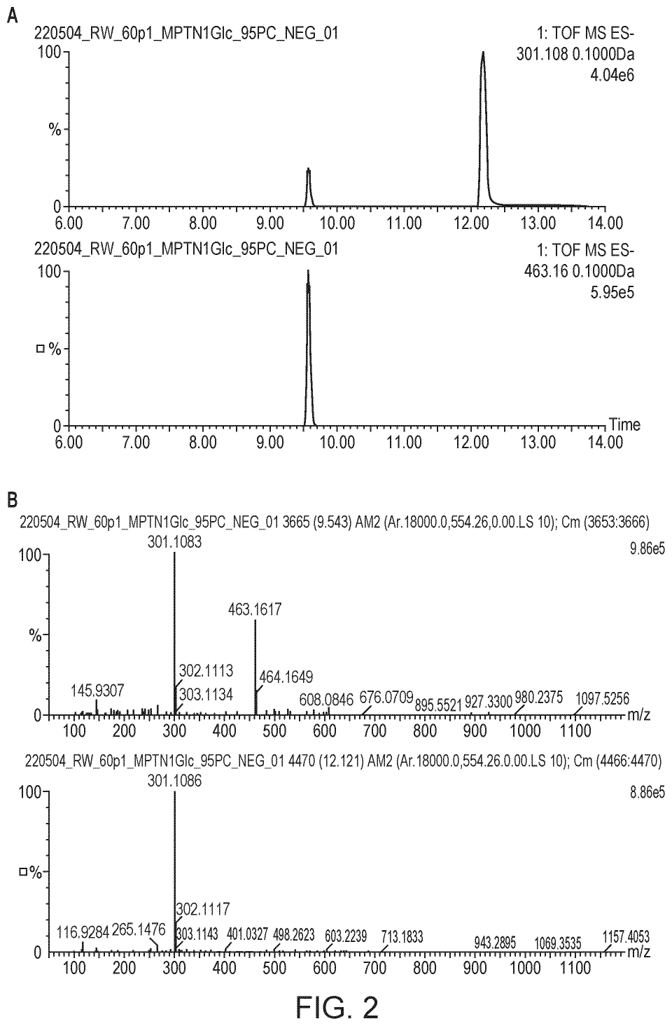

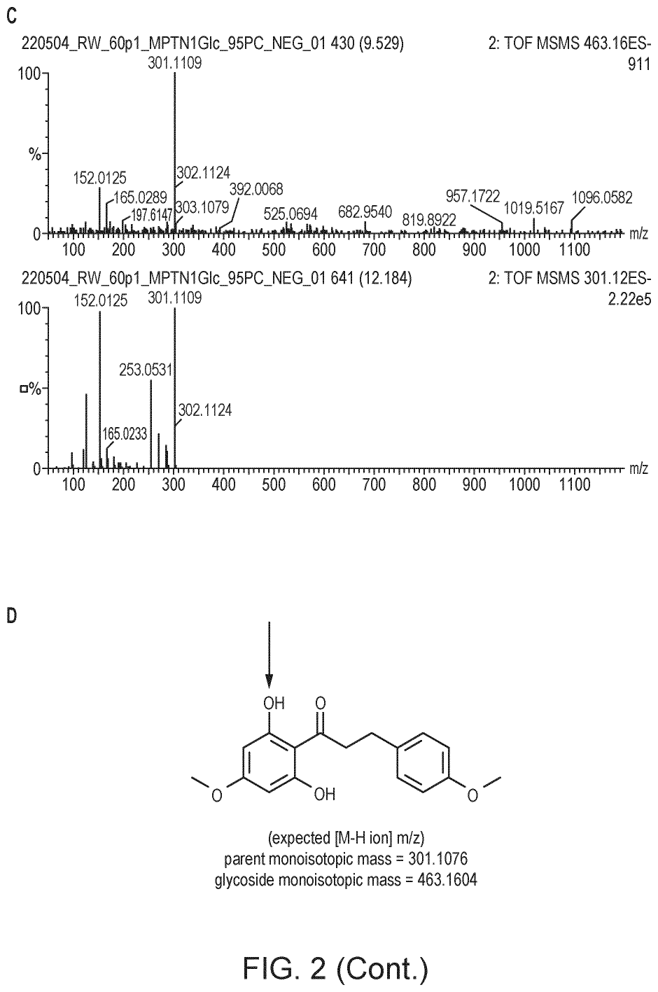

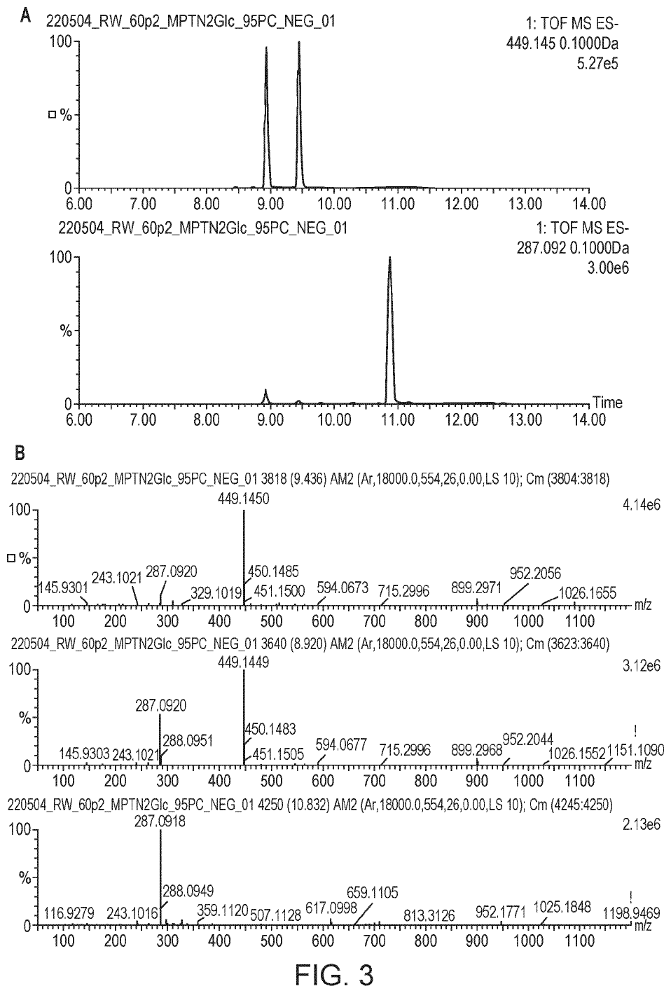

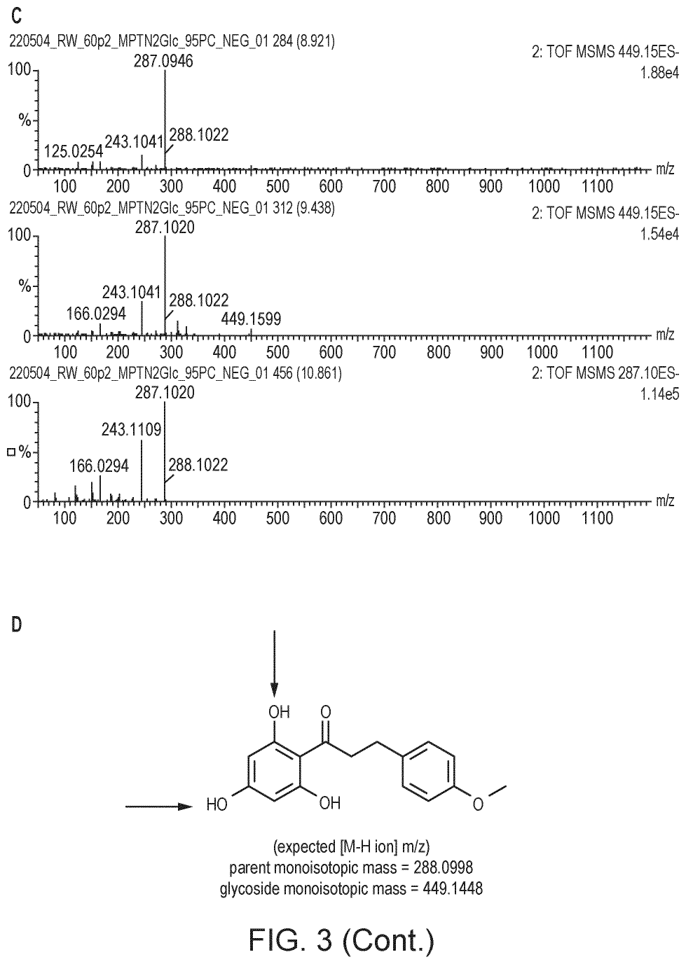

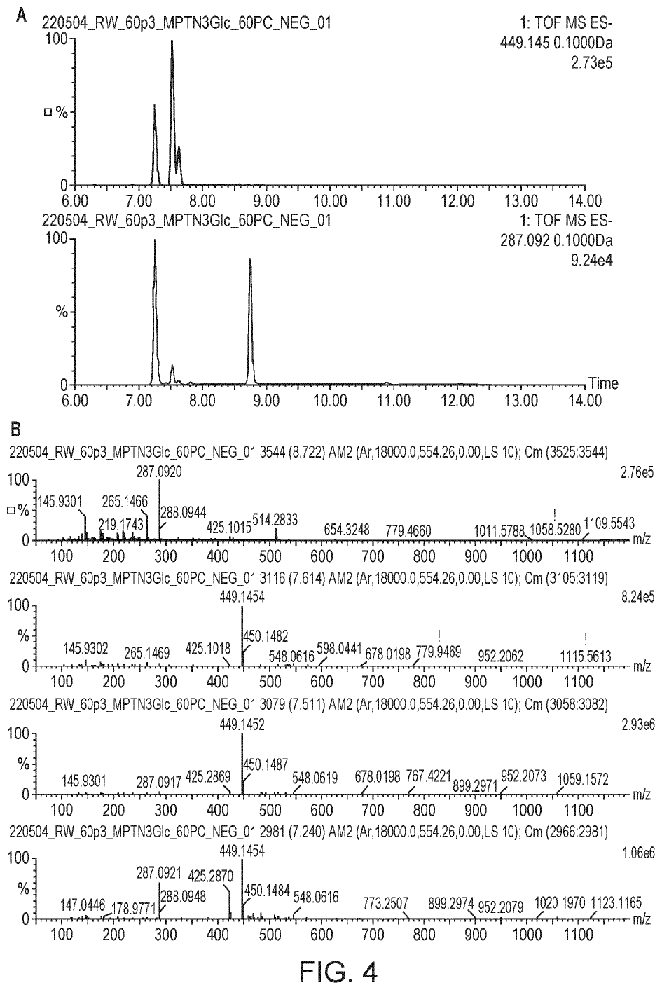

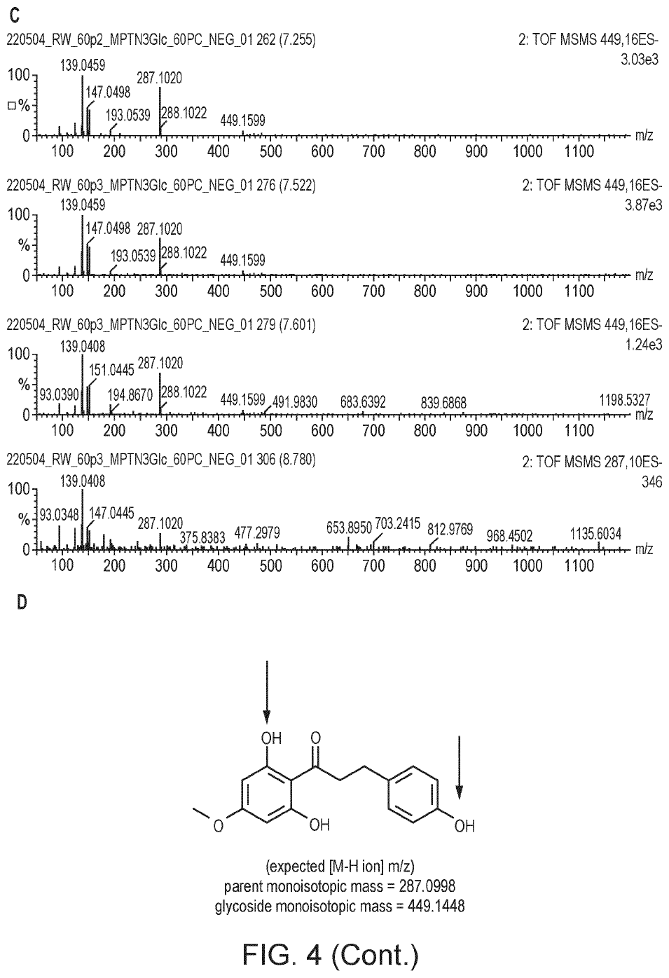

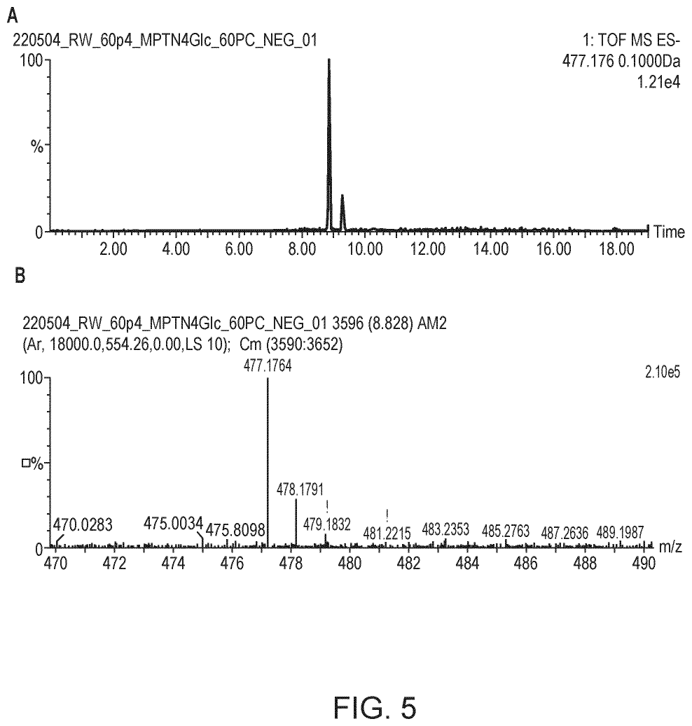

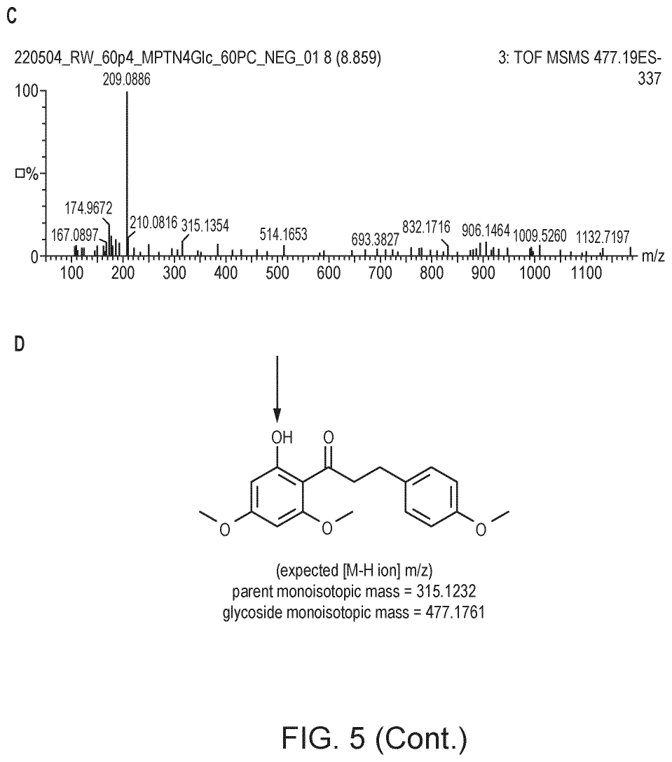

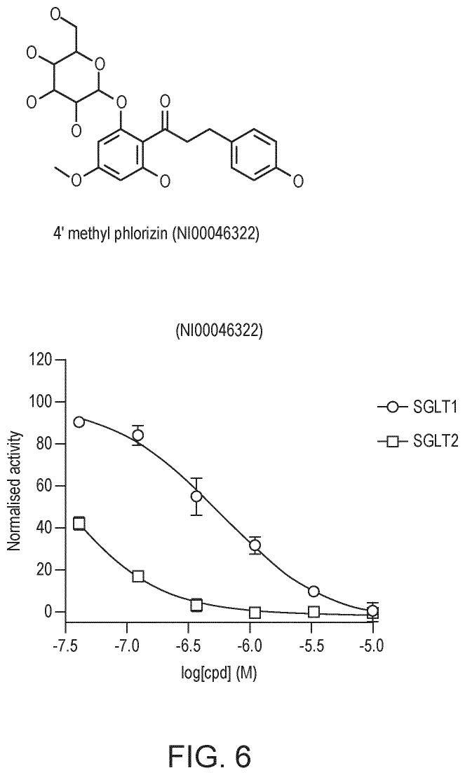

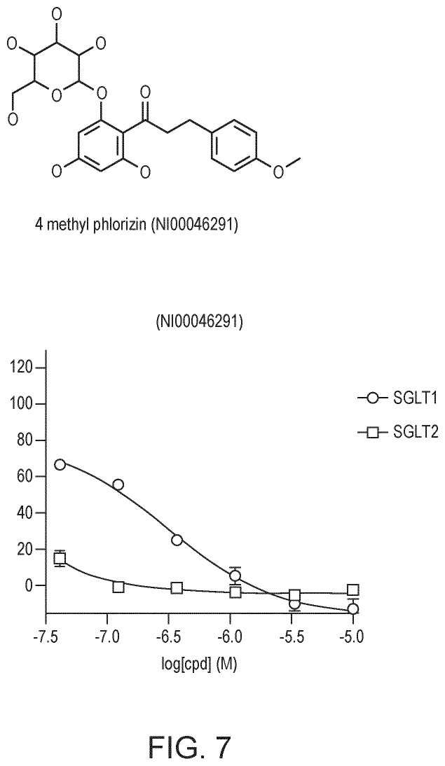

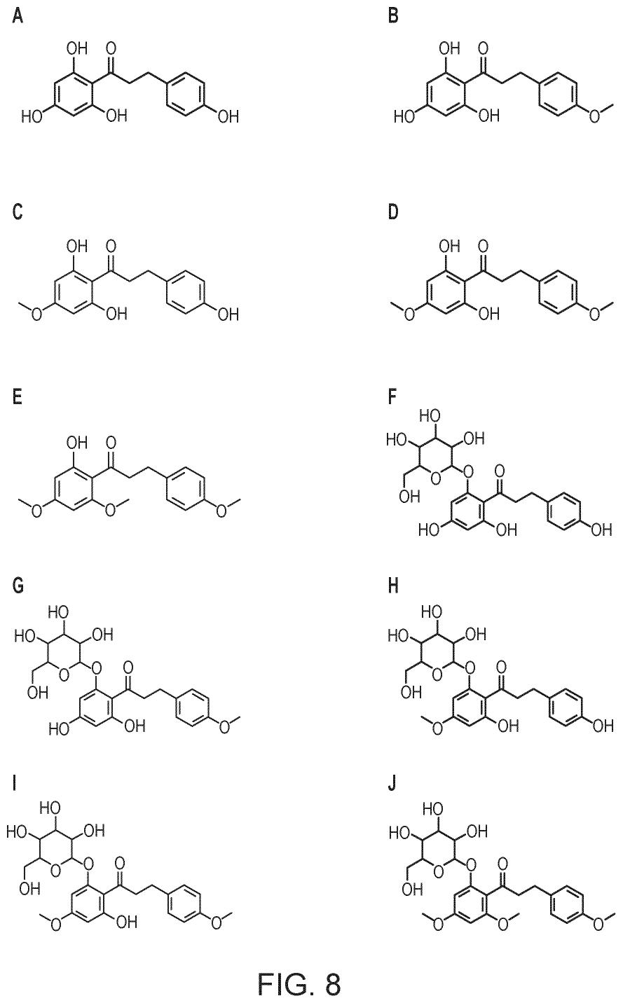

Data presented in FIG. 1 shows level of incorporated [3H]2-deoxyglucose, a GLUT transporter substrate following treatment with (A) Phloretin, (B) Calomelanone, (C) Phloretin 4-methyl ether, (D) Asebogenin, (E) Flavokavain A. Data is shown relative to the effect of the pharmacological GLUT inhibitor BAY-876 (100% effect) and DMSO (0% effect). The data indicates that methylated analogs of phloretin exhibit a reduced degree of GLUT inhibition compared to phloretin.

### Example 2

### Protocol

Methylphloretin compounds were incubated in the presence of rat liver microsomes (M9066-1VL, Sigma) at 1 mg/ml in 50 mM Tris-HCl pH 8.0 containing 5 mM UDP-glucose, 25 mM sucrose, 10 mM MgCl2 and 1 mM DTT for 4 hours with shaking at 150 rpm at 37° C. Samples were prepared for LC-MS analysis by solid phase extraction using Waters Oasis HLB SPE cartridges using a benchtop vacuum manifold. Briefly, the SPE cartridges were activated with at three column volumes of methanol, equilibrated with reaction buffer, sample was applied and washed with deionised water. Analytes were eluted in fractions containing increasing concentration of methanol. SPE eluate fractions were dried under an N₂ stream and reconstituted in 20% methanol. Samples were run on a Waters Acquity uHPLC system with a 10 cm Waters Aquity HSS T3 1.8 μM column held at 40° C. hyphenated to a Synapt G2-S high resolution mass spectrometer operating in negative ion Time of Flight mode. Analysis were performed and analysed using MassLynx 4.2 software. A linear gradient from 0% acetonitrile to 100% acetonitrile was applied over 0-16 minutes. Mobile phases A and B were water and acetonitrile respectively containing 0.1% formic acid. Waters Lockspray technology was applied for the determination of exact mass values. Expected monoisotopic masses were calculated using the molecular mass calculator from the molecular formula.

Results are shown as FIG. 2 to FIG. 5

Incubations of specified individual methyl-phloretin compounds (Calomelanone) (FIG. 2); Phloretin-4 methyl ether (FIG. 3); Phloretin-4′ methyl ether, Asebogenin (FIG. 4); Flavokavain (FIG. 5) with UDP-glucose in the presence of rat microsomes were performed and analysed by uHPLC-MS following solid phase extraction as described.

For each of the above compounds:
Extracted mass chromatograms are shown for the expected monoisotopic masses of the parental methylphloretin and glycoside forms from an LC-MS analysis of an SPE eluate fraction containing both parent and generated glycoside metabolites, with the exception of Flavokavain where the aglycone form was not readily observable in negative ion time of flight mode and only the glycoside form is shown (A).
For each peak observed in the extracted mass chromatograms for parent and glycoside species, a summation of the MS level spectra obtained over the peak duration is displayed (over at least five scans), to which the Waters Lockmass correction has been applied for the determination of accurate masses (B).
For each major peak for parent and derived glycoside species observed in the extracted mass chromatograms, an MS/MS spectra for the parent ion, and the glycoside species is shown (C). For the glycoside species, the selected ions fragment with a loss of 162 mu, consistent with loss of a glucose molecule, to produce fragmentation ions at the expected m/z of the parent methyl-phloretin compound.
For each methylphloretin species, the position of expected non-equivalent sites of glycosylation are indicated (D).

### Example 3

### Assay Protocol:

Briefly CHO-K1 cells stably transfected with SGLT1 or SGLT2 were plated at 20000 cells/well in a 384 plate (Black clear bottom Greiner Bio-One #781091) for 24 hours in 50 ul of culture medium (RPMI1640+5% serum). Prior to the experiment cells were starved for 3 hours by removing culture medium and replacing it by 20 μl of medium without glucose and serum. The experiment was triggered by the addition of 20 μl of assay buffer containing 320 μM of 1NBDG (fluorescent Glucose analogue) in a glucose and serum free medium with or without compounds in 1% DMSO final and incubated at 37° C. for 2 h. The cells were then washed 3 times using PBS to remove any trace of extracellular 1NBDG. Fluorescence (RFU) corresponding to 1NBDG uptake inside the transfected cells was then measured on a multimode reader (Bioteck synergy neo signal exc 485 nm em 535 nm).

Data were finally normalized: percentage activity was determined using DMSO as maximum uptake signal (100%) and 10 μM of dapagliflozin as minimum signal (0% full uptake inhibition) using the formula: (x−min)/(max−min)*100

### Results

In these graphs each compound has been tested at 6 different concentrations starting at 10 μM with serial 1/3 step dilution in duplicate both against SGLT1 and SGLT2 transfected cell lines. In absence of inhibition (DMSO control) the uptake of 1NBDG is maximal resulting in a high RFU signal which is used to determine the 100% normalized activity, at the opposite in presence of 10 μM dapagliflozin there is a complete inhibition of the active transport of 1NBDG, the resulting fluorescence observed in this condition gives the 0% normalized activity: the following formulae is applied for normalization:

$$Normalized activity ( cpd ) = ( RFU ( cpd ) - RFU ( dapa ) ) / ( RFU ( DMSO ) - RFU ( dapa ) ) .$$

FIG. 6 shows normalized SGLT1 and SGLT2 inhibition activities of 4′ methyl phlorizin (NI00046322). The downward direction of both curves represents therefore an increase in the inhibition of SGLT1 and SGLT2 with the increase of the tested concentration of 4′ methyl phlorizin (NI00046322).

We observe that 4′ methyl phlorizin (NI00046322) is active on both SGLT1 and SGLT2 with different IC50s. 4′ methyl phlorizin (NI00046322) is more potent on SGLT2 than on SGLT1 as we observe a complete inhibition at 1 μM (−6 in log scale) with an IC50 of about 33 nM (−7.5 in log scale) for SGLT2 whereas the complete inhibition is observed at 10 μM (−5 in log scale) with an IC50 between 300 nM and 1 μM (between −6.5 and −6 in logscale).

FIG. 7 shows normalized SGLT1 and SGLT2 inhibition activities of 4 methyl phlorizin (NI00046291). The downward direction of both curves represents therefore an increase in the inhibition of SGLT1 and SGLT2 with the increase of the tested concentration of 4 methyl phlorizin (NI00046291).

We observe that 4 methyl phlorizin (NI00046291) is active on both SGLT1 and SGLT2 with different IC50s. 4 methyl phlorizin (NI00046291) is more potent on SGLT2 than on SGLT1 as we observe a complete inhibition at 1 μM (−6 in log scale) with an IC50 of about 33 nM (−7.5 in log scale) for SGLT2 whereas the complete inhibition is observed at 3 μM (−5.5 in log scale) with an IC50 between 300 nM and 1 μM (between −6.5 and −6 in logscale).

1: A method of inhibition of SGLT2, the method comprising administering an effective amount of at least one methylated phloretin analog to a subject.

2: The method according to claim 1, wherein the at least one methylated phloretin analog comprises one or more of Phloretin-4-methyl-ether, 4′-O-Methylphloretin, Calomelanone or Flavokavain A.

3: The method according to claim 1, wherein the at least one methylated phloretin analog is obtained from a plant or plant extract.

4: The method according to claim 1, wherein the at least one methylated phloretin analog is from unripe apple or orange peel.

5: The method according to claim 1, wherein the inhibition of SGLT2 is in muscle and/or kidney tissues.

6: The method according to claim 1, wherein the subject is in need of treating and/or preventing a condition, disorder, or disease related to type 2 diabetes and/or obesity.

7: The method according to claim 1, wherein the subject has or is at risk of a condition, disorder, or disease related to cardiovascular, weight management, renal, kidney, or brain.

8: The method according to claim 1, wherein the subject has or is at risk of a condition, disorder, or disease responsive to SGLT2 inhibition in a subject in need thereof.

9: The method according to claim 1, wherein the at least one methylated phloretin analog is administered in a food, beverage, or dietary supplement.

10: The method according to claim 1, wherein the at least one methylated phloretin analog is administered in a composition that further comprises a pharmaceutically acceptable carrier.

11: A method of treating and/or preventing a condition, disorder, or disease related to type 2 diabetes and/or obesity in a subject in need thereof, the method comprising administering an effective amount of at least one methylated phloretin analog to the subject.

12: A method of treating and/or preventing a condition, disorder, or disease responsive to SGLT2 inhibition in a subject in need thereof, the method comprising administering an effective amount of at least one methylated phloretin analog to the subject.

13: A pharmaceutical composition comprising a therapeutically effective amount of at least one methylated phloretin analog, or a pharmaceutically acceptable salt or solvate thereof, as active ingredient, and a pharmaceutically acceptable carrier, for inhibition of SGLT2.

14: An in vitro method of inhibiting SGLT2, the in vitro method comprising contacting at least one methylated phloretin analog with SGLT2 and determining inhibition of the SGLT2.
